(19) United States
(12) Patent Application Publication
(10) Pub. No.: US 2015/0290351 A1
(43) Pub. Date: Oct. 15, 2015

(54) Volatile Material Dispenser and Dispensing Screen Thereof

(71) Applicant: I&I Developments Ltd., Barnet, Hertfordshire (GB)

(72) Inventor: Brian Parry Slade, Ashford, Kent (GB)

(73) Assignee: I&I Developments Ltd., Barnet, Hertfordshire (GB)

(21) Appl. No.: 14/438,486

(22) PCT Filed: Oct. 22, 2013
(86) PCT No.: PCT/GB2013/052753

§ 371 Date: Apr. 24, 2015

(30) Foreign Application Priority Data
Oct. 24, 2012 (GB) 1219125.0

(51) Int. Cl. A61L 9/12

(52) U.S. Cl. CPC A61L 9/127

(57) ABSTRACT

A dispenser for dispensing volatile material by evaporation comprises a reservoir for containing volatile material to be dispensed. The dispenser further comprises a dispensing screen for dispensing volatile material by evaporation, the dispensing screen comprising a sheet of material having continuous first and second edges and forming a continuous loop of material extending between the first and second edges. Communicating means can carry volatile material from the reservoir to an in use upper edge of the dispensing screen. The material of the dispensing means is acutely angled to an in use vertical axis of the dispenser.

The present invention relates to a volatile material dispenser, particularly, but not limited to a dispenser for perfumes, insecticides, insect repellents, anti-viral/bacterial, decongestant inhalant, pheromone and attractant materials.

### BACKGROUND OF THE INVENTION

Different types of fragrance dispensers are known. Some consist of a piece of material which is impregnated with volatile scent chemicals. However, although such products initially provide high levels of scent delivery, this reduces as the concentration of scent chemicals in the material reduces. Similar disadvantages exist with gel based air fresheners, in which the fragrance material is provided in a gel and evaporates into the air.

In order to overcome such problems, dispensers in which the volatile material is stored in a reservoir and delivered to a dispensing material are known. In particular, so called “plug in” dispensers are available, in which the volatile material is dispensed with the aid of a heated wick to encourage evaporation.

Also, a wick can be used to dispense the fragrance from a reservoir. However, as fragrances generally comprise different “notes”, which evaporate at different rates (“high” notes evaporating more quickly than “bottom” notes), such wicks generally become saturated and clogged with the least volatile “bottom notes” of the fragrance and the carrier material, so that their effectiveness is therefore reduced over time. A fragrance may contain several fragrance components, solvents and residues. The various components provide the character or profile of the fragrance and they have different volatilities ranging from top note (high) to bottom/end notes (low). Historically perfumers have used bottom notes to sustain conventional fragrance products over time because the volatile top notes tend not to last.

Dispensing insecticides, as opposed to fragrances, requires different considerations due to e.g. their different compositions, different volatilities and the loading they produce on a system. As such, systems designed for linearly dispensing fragrances may not also linearly dispense insecticides. Linear dispensing of insecticides is, of course, highly desirable to ensure substantially constant dosage of materials and effectiveness over the life of the product.

The present invention seeks to overcome or ameliorate at least one of the problems associated with the prior art.

### SUMMARY OF THE INVENTION

According to a first aspect of the invention, there is provided a dispenser for dispensing volatile material by evaporation. In embodiments, the dispenser comprises a reservoir, for containing volatile material to be dispensed. In embodiments, the dispenser comprises a dispensing screen for dispensing volatile material by evaporation, the dispensing screen comprising a sheet of material having continuous first and second edges and forming a continuous loop of material extending between the first and second edges. Further, in embodiments of the invention the dispenser comprises communicating means, for carrying volatile material from the reservoir to an in use upper edge of the dispensing screen. The material of the dispensing means may be acutely angled to a longitudinal axis of the dispenser, which may be an in use vertical axis of the dispenser. In an embodiment, the material of the dispensing means may be positioned at an angle α substantially between 15 and 45° to said longitudinal axis of the dispenser. In alternative embodiments, the angle may be up to about 60° or 70°, or down to about 5° or 10°. In embodiments, angle α may be substantially 5, 10, 15, 20, 25, 30, 35, 40, 45, 50, 55, 60, 65, or 70°. The angle α is defined as the angle between the longitudinal axis and an axis extending along the direction of the minimum distance between a point on the first edge and a point on the second edge.

The longitudinal axis may be a rotational axis of symmetry. The acute angle means that the material of the dispensing means has a radial component and a longitudinal component relative to the axis. The radial separation of the material from the axis may increase all around the loop of material relative to the axis in cross sections moving along the axis.

The dispensing screen may be mounted around the reservoir. This provides a compact package as well as placing the top edge of the dispensing screen close to the top of the reservoir.

The screen may be substantially circular in, in use, horizontal cross section. The diameter of the screen may increase from the top to the bottom of the screen. Alternatively, it may decrease. Further a combination of increase and decrease in diameter may be provided in the horizontal cross section of the screen from top to bottom. The screen may be conical, frustoconical or trumpet shaped, or be comprised of more than one conical, frustoconical trumpet shape joined together. The screen may not be circular in horizontal cross section, but extend laterally by a greater amount in one closed notional ring than at another closed notional ring which is substantially parallel thereto.

The dispensing screen may be mounted concentric to the in use vertical axis of the reservoir, which also provides a compact package for the dispenser.

The dispensing screen may be formed from a sheet of material. Diverting means may be provided on the sheet of the screen, which creates a minimum path length along the surface of the sheet, which is longer than the shortest distance between the first and second edges. The sheet may be woven.

The communicating means conveniently comprises a wicking element, extending from inside the reservoir to the first edge of the dispensing screen. More than one wicking elements may be arranged between the reservoir and the screen, the wicking elements being in communication with one another to deliver volatile material from the reservoir to the screen.

The height of the dispensing screen may be the same or less than the height of the reservoir, providing a compact package and aiding with provision of a hydrostatic head.

A second aspect of the invention provides a dispensing screen for dispensing, by evaporation, volatile materials applied thereto comprising a sheet of material. In embodiments of the invention, diverting means is provided, formed in or on the sheet. In embodiments of the invention, the diverting means form a minimum path length of the sheet for volatile material flowing along it between at least a portion of a first edge and an opposing second edge of the sheet, which minimum path length is longer than the shortest distance between the first and second edges along the surface of the sheet. In embodiments of the invention, the first and second edges of the sheet are each joined so that each is continuous, and the sheet is formed into a continuous loop of material between them. In embodiments of the invention, a first closed notional ring on the surface of the material has a first length around its circumference and a second closed notional ring on the surface of the material, substantially parallel thereto and separated therefrom, has a second length around its circumference, the second length being longer than the first length.

In embodiments of the invention, the area of the cross section through the first ring is smaller than the area of the cross section through the second ring. In embodiments, the minimum distance from a notional axis through both rings of the first ring is smaller than the minimum distance from the same notional axis of the second ring.

One edge of the sheet may be longer than the other. The second edge may be longer than the first edge. At least one of the rings may be positioned between the first and second edges.

The material of the dispensing means may be oriented at an angle α substantially between 15 and 45° to said notional axis. In alternative embodiments, the angle may be up to about 60° or 70°, or down to about 5° or 10°. In embodiments, angle α may be substantially 5, 10, 15, 20, 25, 30, 35, 40, 45, 50, 55, 60, 65, or 70°. The angle α is defined as the angle between the notional axis and an axis extending along the direction of the shortest distance between a point on the first edge and a point on the second edge.

The screen may additionally comprise one or more supports or mounts, which hold the sheet in a predetermined shape/configuration. The sheet may be held in shape by such a support at or adjacent each edge of the screen. The supports may be substantially parallel.

The sheet may be formed of a woven material. The material may be stretchable and resilient. The material may be tensioned, for example by the supports by which it is held in shape. The sheet may have a minimum extent laterally from a notional axis extending between the two edges away from one of the edges, which is larger than the minimum lateral extent from the notional axis at each of the edges. A further support may be provided between the edges of the sheet, to hold a ring of the sheet to a larger minimum distance laterally from the notional axis than at least one of the edges. The supports may be generally circular.

The sheet of material may be substantially conical or frustoconical.

The first ring and the first edge may be coincident, the second ring and the second edge may also or alternatively be coincident. Alternatively, the first ring and the second edge may be coincident. The first and/or second rings may be located between the first and second edges. The first and second rings may be substantially circular, oval, square, or any other polygonal shape. A notional ring of the material between the two edges may have a minimum lateral extent from the notional axis between the edges more than that at each edge.

In general, the volatile material is applied to the dispensing screen in a liquid carrier at the first edge, and the volatile material in the liquid carrier flows towards the second edge by capillary action, gravity or a combination of both, evaporating as it flows. In embodiments where gravity is used to propel the flow of material down the sheet, the convoluted path may reduce the apparent effect of gravity by preventing a fully vertical flow of the material down the sheet. The gravity flow removes the need for a power source to pump material. Further, application of fresh volatile material to the top of the sheet in embodiments washes any residue from previously applied material down the sheet to reduce clogging and the consequent reduction in performance. The sheet is effectively irrigated by newly applied carrier liquid and volatile material to “wash” down already applied material and keep the sheet clear for carrying more material for evaporation. The convoluted path increases the path length for any given size of sheet. The sheet can therefore be made more compact than would otherwise be possible, and extra structural features to support the sheet can be reduced. These factors may also serve to reduce cost of manufacture of the sheet.

Such a screen can produce constant or near-constant evaporation of the volatile material, and also consistent ratios of the different chemicals in the volatile material over time. Therefore the odour intensity, and the particular scent, do not substantially change over the lifetime of the dispenser. In the case of an insecticide, insect repellent, anti-viral/bacterial, decongestant inhalant, pheromone or attractant material use, the dispensing is constant, so the dosage of materials released is also substantially constant.

The reservoir may be as shown in U.S. Pat. No. 7,360,671 or U.S. Pat. No. 6,631,891, the entire contents of each of which are incorporated herein by reference. In this case, a reservoir is provided, in which volatile material is contained. A wick extends substantially vertically down into the reservoir. The sheet may be positioned relative to the reservoir to allow siphonic feeding of the volatile material to the top of the sheet, at the first edge. Alternatively, and preferably, the wick may be fed by the constant hydrostatic head provided by the pressure compensated reservoir disclosed in U.S. Pat. No. 7,360,671. The effective constant height of the bottom of the reservoir when using the dispenser described in these two documents provides a substantially constant flow rate of volatile material to the top of the sheet.

Capillary and gravity forces combine together to load the dispensing sheet. Gravity becomes more dominant as the capillary force diminishes as the sheet becomes loaded. Gravity acts vertically downwards on each molecule of liquid in a column, singularly and collectively. Therefore, a straight path, which was aligned vertically, may make the liquid flow down the sheet too fast to provide sufficient time for evaporation.

It can be seen that the path taken by a single molecule on the sheet with a herring bone pattern/sexangular mesh fabric is convoluted. As it travels from the top to the bottom of the screen along the ‘convoluted path’, the distance is greater than the actual vertical length of the screen, ie the shortest length along the surface of the screen from the first edge to the second edge. The evaporating capacity may be directly proportional to the surface area of the, or each, screen. The convoluted path may comprise a plurality of fluid pathways. The number of fluid pathways may change between the first edge and the second edge of the sheet. The number of fluid pathways may increase from the first edge to the second edge of the sheet.

The other effect is gravity, acting on each liquid molecule singularly and the whole column collectively. Most of the path taken by the liquid column is disposed at an angle (inclined plane) to the vertical. This slows down the effect of gravity acting on the column of liquid. The provision of perforations in the surface of the sheet makes it highly permeable and therefore very sensitive to any small movement of surrounding air. Further, in the case of a woven material such as a sexangular mesh fabric, the tension applied to the opposing edges will affect the speed of travel of the liquid on the sheet. In particular, with higher tension, the spaces are stretched in one direction, and tend to be squashed perpendicular to that direction. Thus, the degree of convolution of the woven fibres from the opposing edges is reduced. Even if the sheet is not squashed by the applied tension in a direction perpendicular thereto, the portions of the woven material that form the sides which are neither horizontal nor vertical will be made closer to vertical, and gravity will therefore pull the volatile material down the sheet more quickly. The material can be highly permeable to air, due to the high surface area of the strands relative to the surface area of the sheet if it were solid. The strands may be fine polyester monofilaments, which may be woven into the sheet of the screen. The material may be self irrigating.

It can also be seen that the convoluted path influences the fragrance material to encircle each and every hole in the surface of the sheet when travelling from the top of the screen towards the bottom. Capillary forces combine with gravity in helping to distribute the fragrance uniformly across the sheet's surface.

In embodiments, the wick feeds the sheet by siphonic action. However, alternatively, where a liquid has a low volatility and a low viscosity is used, siphonic action may not be used. An example is the liquid EXXSOL D 40, ISOPAR-L and ISOPAR-M which is used as a carrier for an ingredient to kill mosquitoes. In the present embodiments the active ingredient is in a low concentration between approx 0.5 and 2%. Preferably, the concentration is between approximately 0.89 and 1.78%, as this has been found to be effective. It will, however, be appreciated that other concentrations could be employed, and particularly if a different carrier is used. Other possible carriers could be used subject to testing of volatility and performance. These are: EXXSOL D80 and EXXSOL D100.

How far the liquid travels around the fabric circuit depends upon the temperature. As the temperature increases, there is an increase in the volatility of the liquid product and therefore the liquid evaporates at a faster rate and will only travel a relatively short distance along the pathway. When the temperature is lower, the volatility of the liquid is reduced and evaporated at a lower rate. Therefore the liquid, on average, travels to a point further along the pathway before evaporating. The temperature compensation effect can be seen as the result of a higher volatility of product dispensed from a smaller surface area equates to a lower volatility of product evaporated over a larger surface area.

Aspects and embodiments of the invention are therefore, for the various reasons given above, particularly suitable for dispensing low volatility liquid insecticides.

### DETAILED DESCRIPTION OF EMBODIMENTS OF THE INVENTION

Embodiments of the invention will now be described, purely by way of example, with reference to the accompanying figures, in which:

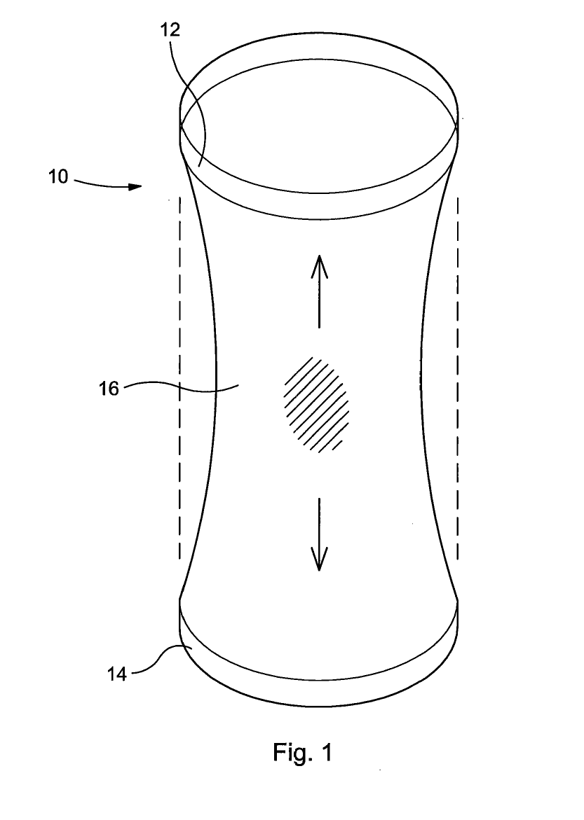

FIG. 1, discloses a dispensing screen comprising a cylindrical sheet of material that has been stretched from its cylindrical ends longitudinally;

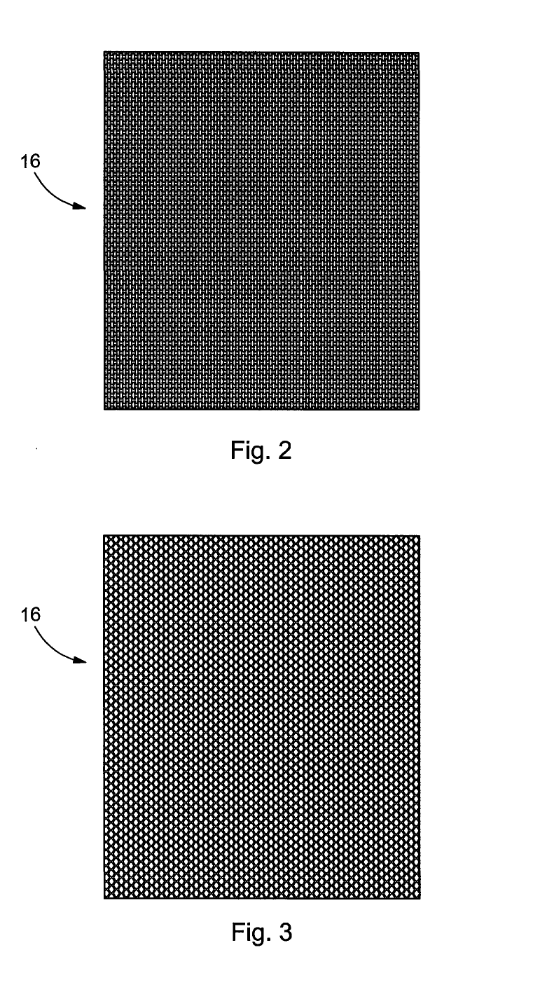

FIGS. 2 and 3 show the effect of this longitudinal stretching on a sheet of woven material comprising the screen;

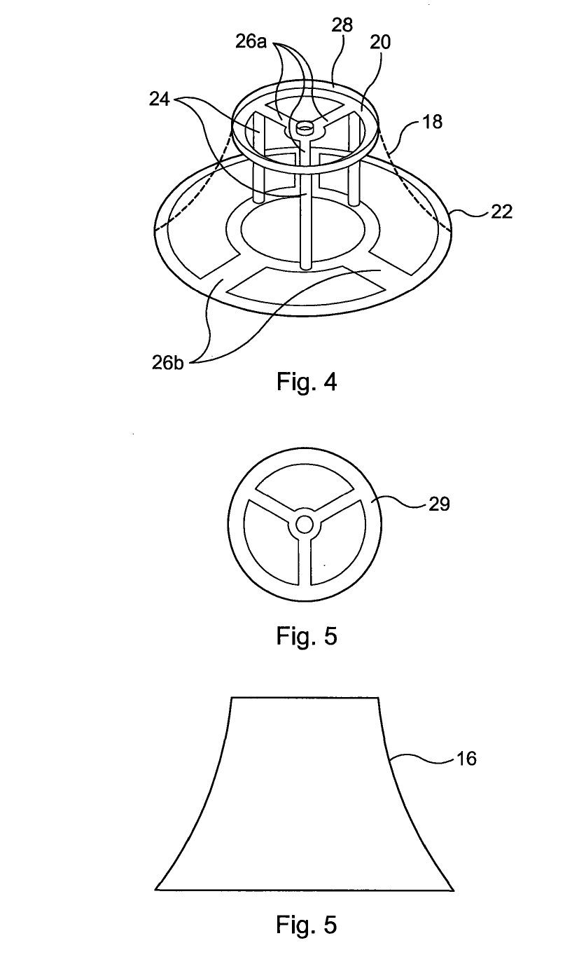

FIG. 4 shows a dispensing screen according to a first embodiment of the invention;

FIG. 5 shows a cross section at one end of the screen shown in FIG. 4;

FIG. 6 shows a longitudinal cross section of the screen of FIG. 4 through a notional axis;

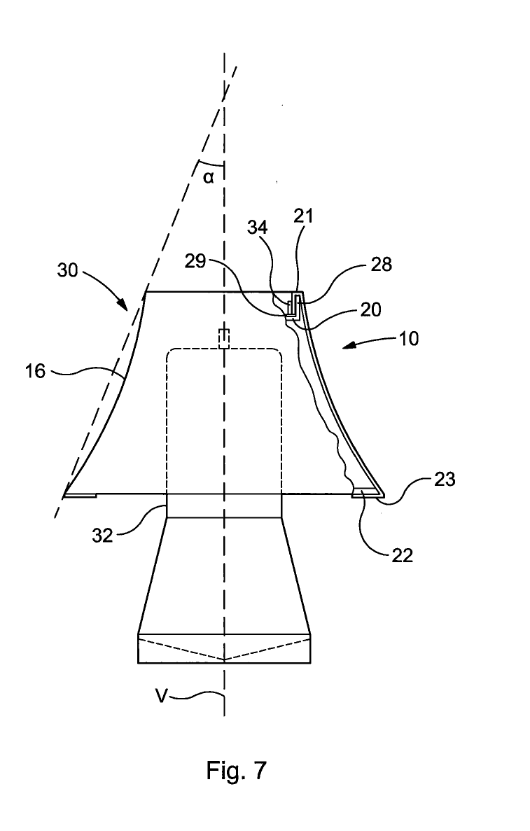

FIG. 7 shows a dispenser according to a second embodiment of the invention incorporating a dispensing screen according to the first embodiment of the invention;

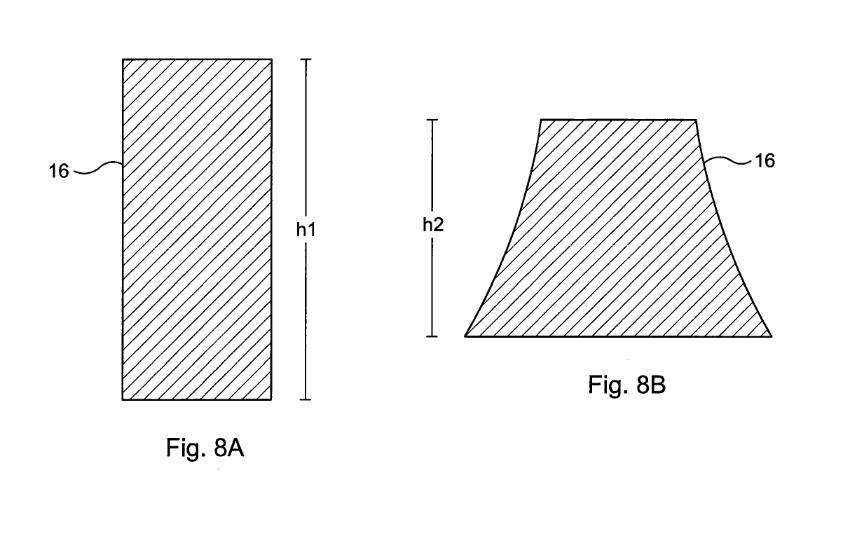

FIG. 8 shows the effective heights required of cylindrical and frustoconical screen shapes to achieve the same surface area dimensions;

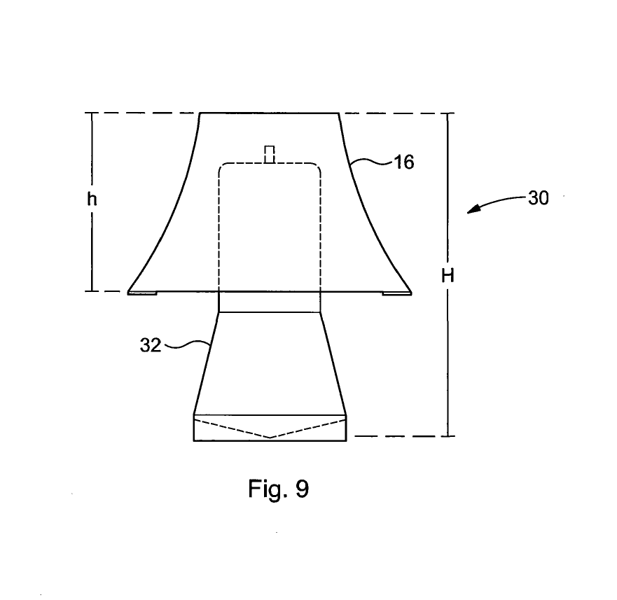

FIG. 9 shows, schematically, the relative heights of the dispensing screen and the dispenser of FIG. 7;

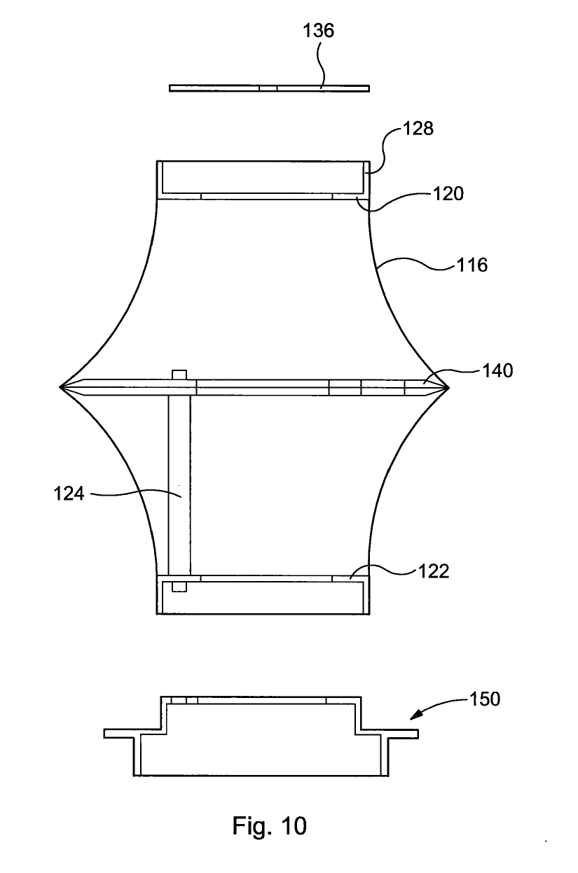

FIG. 10 shows a dispenser according to a third embodiment of the invention;

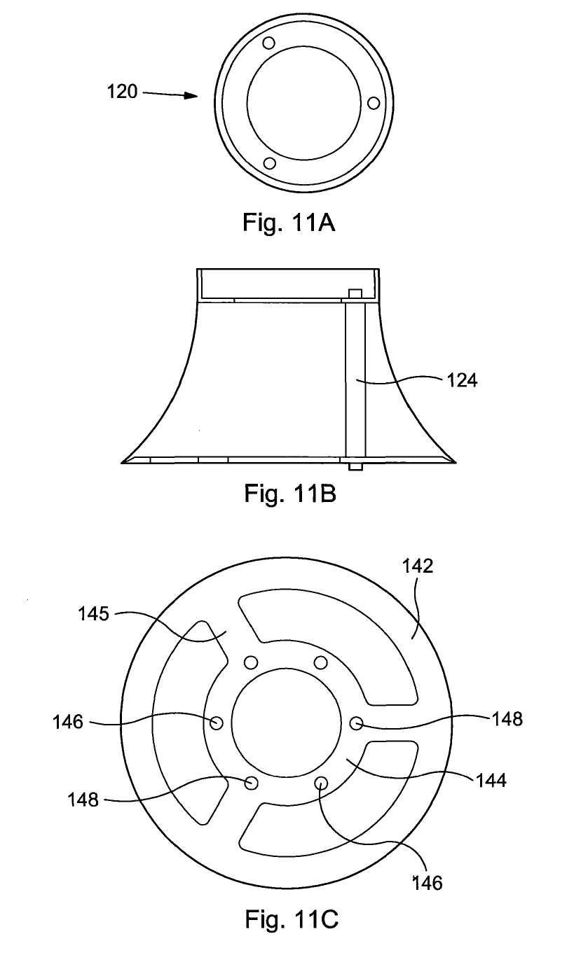

FIGS. 11a, b, and c show a modification of the dispensing screen shown in FIG. 7;

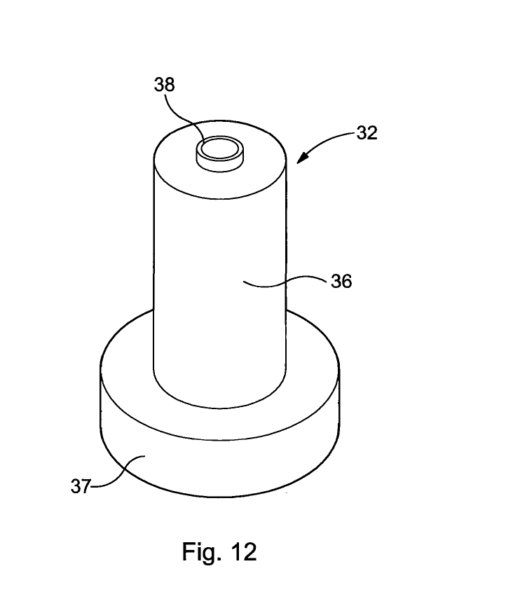

FIG. 12 shows a reservoir for use in embodiments of the present invention;

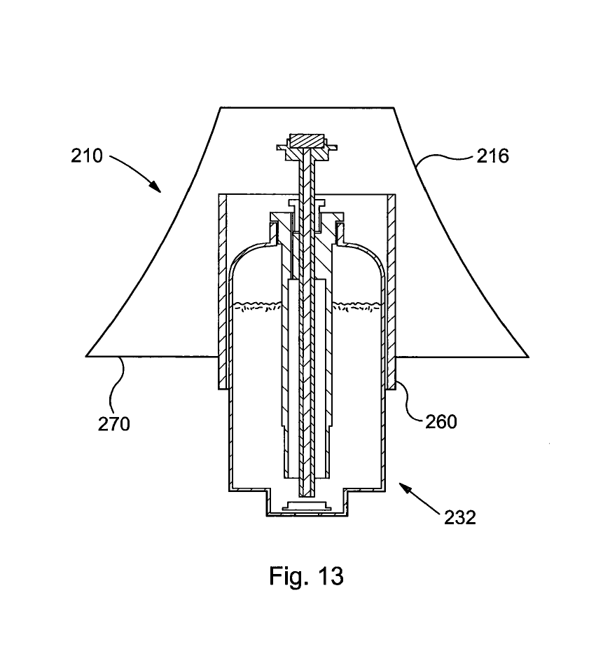

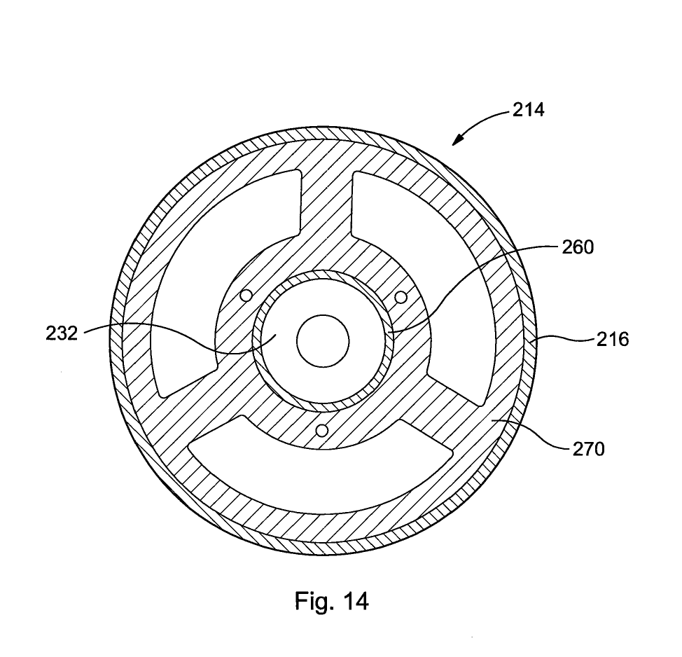

FIGS. 13 and 14 show a dispenser according to a further aspect of the invention; and

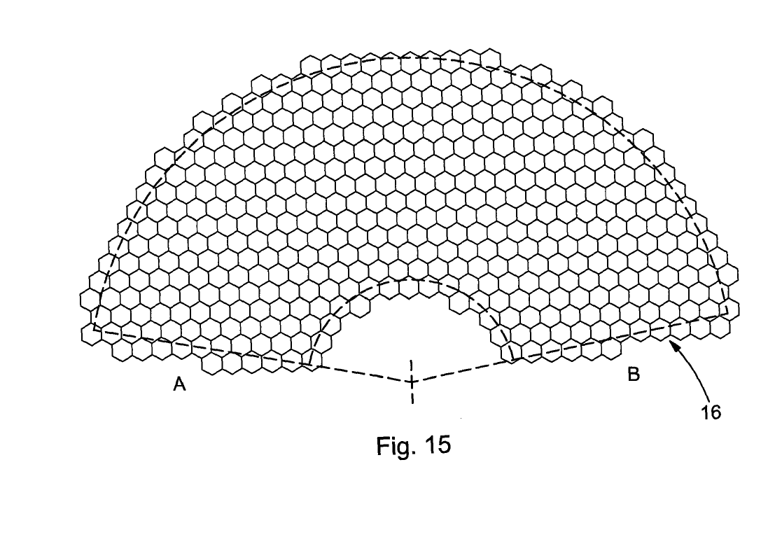

FIG. 15 shows a sheet for use in embodiments of the invention.

Turning now to FIGS. 1 and 2, FIG. 1 shows a dispersal screen 10 comprising two circular supports 12, 14, at the bottom and the top. A sheet of material 16 is provided, extending between the circular supports 12, 14. The sheet 16 of the present embodiment is formed from a flexible, stretchable, and resilient woven material, having a high permeability to air, so it is necessary for it to be tensioned lengthwise between the top and the bottom as indicated by the arrows, in order that it maintains its shape.

In the present embodiment the material is Litmans 573, which has a low loading capacity for volatile liquids. The material has a high capillarity, but low absorbency. To provide good permeability, the sheet has a high void ratio and when it is pulled taut from the top to bottom to secure it to the support, these holes in the fabric structure become elongated. However, this produces the effect shown in FIG. 1 where a waist is formed in the wall of the sheet of the dispersal screen. Litmans 573, being a woven or mesh material, made from polyester, is particularly useful for emanating insecticides, which are typically low volatility liquids. Litmans 573 provides high exposure of surface area for the emanation. Using polyester is advantageous due its resistance to solvents and the smoothness of its surfaces that aids its capillarity.

FIG. 2 (for clarity on a black background) shows how the hexagonal holes in the woven tensioned material of the sheet 16 in FIG. 1 become deformed by becoming elongated when subjected to tension in a single direction, in this case along the cylindrical axis. This shows the deformation of the cylindrical sheet. There are several adverse effects of this: 1) the fibre density per square centimetre is increased; 2) the permeability to air is reduced; 3) the grain of the pattern is vertically biased; 4) the loading of liquid on the screen is increased; 5) the increase in saturation of liquid on the sheet 16 reduces the effective evaporative surface of the woven fabric of the sheet.

In FIG. 3 (again for clarity on a black background) it can be seen that this sheet is evenly tensioned between the cylindrical axial and circumferential directions. The configuration in FIG. 3 has a lower fibre density than that shown in FIG. 2 and greater permeability to air. This will have a lower loading of liquid and as a result have a higher evaporative surface area. Further, because the elongation is no longer only along the cylindrical axis, the angle of each of the weaves extends to a greater degree across the cylindrical axis, so increasing the minimum path length for volatile material travelling down the cylinder, and slowing the rate of descent of the volatile material when it is applied to one cylindrical end of the screen, which is orientated with its cylindrical axis substantially vertically, and moves move slowly down the material.

FIG. 4 shows a means for achieving the configuration of the sheet 16 shown in FIG. 3. A part conical support structure for the sheet is provided and, although the tension is applied between the top and the bottom of the structure (as in the case of the cylinder in FIG. 1), a very different result is provided, due to the angle produced by the two diameters and the distance between them. The support structure of FIG. 4 provides a material structure substantially according to FIG. 3. The curved dotted lines 18 indicate how the woven fabric forms around the structure. The structure is formed of two concentric and parallel circular supports 20, 22, which are separated from one another. The upper support 20 is of smaller diameter than the lower support 22. Holding the structure together and maintaining the separation of the upper and lower supports 20, 22, are three rods 24. The rods may be joined with the supports by any convenient method. With this arrangement tension can be applied to the material as the rods 24 counter the compressive force between the upper and lower supports 20, 22. The upper and lower supports 20, 22 are formed substantially open, so as to improve air flow around the screen 10.

In the present embodiment, the supports 20, 22 are each formed of two concentric, coplanar, annular rings, joined by three radial braces 26a, 26b. The upper support 20 comprises a lip or flange 28, which allows a wicking element, not shown, to be placed in the recess formed thereby to transport the volatile material from a central hole in the upper support 20, in which is placed a porous stud, and to which a reservoir holding the volatile material (not shown) is coupled, to the outer circumference of the upper support 20, to which the sheet 16 is coupled. The sheet 16 is coupled to the support and the wick via known methods. A ring is provided (not shown), mounted over the wick and sheet and attached to the upper support, by a friction fit. Alternatively, adhesive or mechanical means may be used to secure the ring, for example.

FIG. 5 shows a stamped out wick 29, in the present embodiment in the form of a paper element that connects between the porous stud in the middle at the top of the upper support and the top of the sheet. It fits inside the recess at the top of the support 20 and is retained in contact with the top of the screen by a ring (not shown).

FIG. 6 shows a profile (side view) of the screen, comprising the sheet 16 fitted to the frustoconical support structure shown in FIG. 4. In this embodiment, the diameter of the lower support 22 is twice the diameter of the upper support 20.

FIG. 7 shows the dispensing screen 10 with the reservoir/cartridge 32 installed to form a dispenser 30. The cutaway section shows how the sheet 16 is secured to the upper support 20, via the ring 34. The porous wicking element 29 is also shown. The first (upper) edge 21 of the sheet is mounted on the upper support 20 as discussed above. The second (lower) edge 23 of the sheet 16 is glued to the lower support 22.

The sheet 16 is mounted on the circular supports 20, 22 and thus has a substantially circular cross section substantially normal to the axis of the screen. A first closed notional ring on the surface of the material of the sheet 16 has a first length around its circumference and a second closed notional ring on the surface of the material, substantially parallel thereto and separated therefrom, has a second length around its circumference, the second length being longer than the first length. The notional rings in the present embodiment are substantially normal to the axis of the screen. It can be seen that the area of the cross section through the first ring is smaller than the area of the cross section through the second ring. The minimum distance from a notional axis through both rings, in the present embodiment the rotational axis of symmetry of the screen, of the first ring is smaller than the minimum distance from the same notional axis of the second ring. In the present embodiment, the first notional ring is a first edge 21, at or adjacent the upper support 20. The second notional ring is a second edge 23, at or adjacent the lower support 22.

The configuration means that the sheet of material is angled at an angle α to an in use vertical axis (V) of the dispenser along the material from the first to the second edge. In the present embodiment, the angle changes with the position along the axis, but remains acute. FIG. 7 shows how angle α is defined as the angle between the vertical axis (V) and an axis extending along the direction of the minimum distance between a point on the circumference of the first support and a point on the circumference of the second support. In other embodiments, the angle α may remain constant along the length of the sheet 16 from the first edge 21 to the second edge 23. In either case, the angle α may be substantially between 15 and 45°, e.g. typically 30°. It has been found that an angle within this range is sufficient to provide the advantages previously described, without extending too far in a direction perpendicular to the vertical axis V, which would be undesirable when the products are placed on shelves for retail. It will, however, be appreciated that other angles may be employed, e.g. up to about 60° or 70°, or down to about 5° or 10°. That is to say, the angle α may be substantially 5, 10, 15, 20, 25, 30, 35, 40, 45, 50, 55, 60, 65, or 70°.

The mesh screen 10, by being formed into a cone or cone-like shape, provides a progressive increase in structural routes to the path of the liquid product. This effect, combined with the screen's high evaporative ability maintains an even loading of liquid over the fibrous structures surfaces of the screen 10 from the top to the bottom. If the screen 10 was formed into a cylinder, the loading at the bottom would be higher than the upper part of the screen.

FIGS. 8 A and B show how a cylindrical sheet has to be much longer than a frustoconical sheet in order to be equivalent in surface area dimensions. A ramification of this (apart from other adverse effects that were mentioned earlier) is the saturation of the material that would be caused by the head height of a column of liquid located in the sheet's woven fabric structure. It can also be seen that the frustoconical configuration is much shorter, and therefore vertically compact, than the cylindrical configuration.

FIG. 9 illustrates the height difference between the reservoir 32 and the sheet of the dispenser 30. Firstly it can be seen from the drawing that the supply system is hydrostatically negatively biased. This is illustrated by the large differences in height between ‘h’ and ‘H’. This difference is much larger than a cylindrical emanating system with the same surface area, as shown in FIGS. 8 A and B. In embodiments of the present invention, flow is thus controlled by the negative hydrostatic force of the fluid circuit that is created by the different heights h and H. Once the fluid circuit is primed by capillary action, the negative hydrostatic force attenuates the flow against which the capillary circuit forces pull.

The resilience of the material in the sheet 16 causes the material to curve radially inwardly. This curvature of the sheet provides a changing angle of the sheet to the vertical, which can be beneficial. This changes the effect of gravity along the axial extent of the screen, ie vertically, in use. As the liquid product travels down the sheet 16, its increase in head height is less relative to its increase in surface area, unlike a cylindrical support, where the head height increases at the same rate. The sheet 16 discriminates between the carrier and the active ingredient by the higher volatility carrier mostly evaporating from the radially smaller upper part of the sheet 16, and the lower volatility active material mostly evaporates from the radially larger lower part of the screen.

FIG. 10 shows an alternative embodiment, where the support structure comprises three supports 120, 122, 140, rather than simply an upper and a lower support. Once again the supports 120, 122, 140 are axially separated, parallel and concentric. However, the upper and lower supports 120, 122 now have the same radius, while a central support 140 is placed axially between them. The upper support 120 is formed in a similar manner as described above, including the wicking element 136, ring (not shown) and lip or flange 128. However, as shown in FIG. 11A, the inner concentric ring and radial braces are not provided in this embodiment. In the present embodiment, the lower support 120 comprises a ring (not shown), similar to the ring associated with the upper support, although without the need for a porous wicking element. Alternatively, the sheet 116 may be glued or otherwise attached to the lower support. The central support 140 is maintained in position, in the present embodiment substantially mid way between the upper and lower supports, by rods 124, in the same way as in the earlier embodiment. Only one of the rods 124 is shown in the Figure for clarity. Although not shown, the central support also has the same structure as the upper and lower supports. The central support 140 comprises two concentric, coplanar annular rings, the rods 124 being attached to the inner ring, and maintaining the separation between the upper and lower supports. This structure allows air movement within the axial extent of the sheet.

A base 150 is provided, on which the lower support 122 is held to hold the dispensing screen in place. In the present embodiment, the lower support also comprises a lip or flange, into which the base is placed. The rods 124 connecting the central support 140 and the lower support 122 extend through the lower support 122 and engage with corresponding holes in the base 150.

FIGS. 11 A, B and C show components of the dispenser of the second embodiment. The central support 140 comprises a pair of coplanar, concentric annular rings 142, 144, connected and held in relative position by three braces 145. Holes 146, 148 are provided for receiving the separating rods 124. The holes alternately receive rods between the central support 140 and the upper support 120 and the lower support 122.

FIG. 12 shows a reservoir according to embodiments of the present invention. As described above, the reservoir is as described in U.S. Pat. No. 7,360,671. The reservoir 32 comprises a body 36, in which the volatile material is contained, a base 37, and a dispensing piece 38 at the top, which houses a wick and a porous stud to conduct the volatile material out of the reservoir 32 and which engages with the porous material 34, which allows communication of the volatile material to the sheet 16. The dispensing piece comprises a circular part, which engages with the inner circumference of the inner ring of the upper support, in order to mount the upper support, and thus the screen, on the reservoir, which also serves as the base of the dispenser.

FIG. 13 shows an alternative dispenser according to embodiment of the invention. The screen 210 is similar to the screen described above, and the reservoir 232 is as described above. However, in this embodiment, the lower edge of the sheet 216 is placed in fluid communication with a sink 260, ie material that absorbs any unevaporated material that reaches the second edge of the sheet 216. Upper and lower supports (not shown) are also provided in the same way as described above. Such an absorbent sink is particularly useful when the volatile material comprises one or more fragrances, where residues may remain, rather than all of the material supplied by the reservoir evaporating from the sheet, as is the case with some insecticides for example. One insecticide that could be used with embodiments of the invention is SumiOne. This may be used as the active ingredient, this being volatile at room temperature, with e.g. ISOPAR M as a carrier.

FIG. 14 shows a view from underneath the dispenser of FIG. 13. Extending between the sheet 216 and the sink 260 is a sheet of paper 270. The sink 260 is wrapped around and secured to the reservoir 232. The base of the reservoir 232 is not covered with the sink 260, so that a user can see when the reservoir is empty and replace then reservoir 232 and sink 260 together as a single unit. The sheet 216 is mounted on the lower support as described above. The paper 270 is mounted on the lower support and has the same shape, and provides a fluid communication path from the second edge of the sheet 216 to the sink 260. Where the paper 270 connects to the sink 260 a flange is formed on the paper 270 to ensure the communication for fluid from the paper 270 to the sink 260. The positioning of the sink 260 is higher than the constant level in the reservoir 232 so gravity has little effect. Further, the sink 260 functions by capillary action upwards so that it deposits the residues at the top first. It has a uniform cross sectional area. Also, although its primary purpose is to filter and collect the residues, it is also a good emanator for the components of the dispensed material with lower volatility, so that more of the product can be dispensed and less is wasted. A base (not shown) is provided on which the reservoir sits, to provide stability to the dispenser.

FIG. 15 shows a manner of forming the screen according to all of the above embodiments. A flat sheet of material is cut into a section of an annular disc of fabric. For clarity, the size of the weave in the fabric is exaggerated many times in the figure. The straight ends of the section A, B are then joined to form a generally frustoconical shape, which may be curved from top to bottom, with the two arcs forming the first and second edges of the sheet 16. As the liquid travels down the sheet 16, the number of liquid pathways increases, progressively reducing the loading of liquid on the screen. No stretching force may be applied to the sheet, which may therefore have a frustoconical shape. Alternatively, the first and/or second edges may be stretched, so that the shape is as shown in FIG. 9 or 10 for example.

As a further alternative, a rectangular piece of material, or a sheet which is formed in arc, but not circular arcs, may be used as the base to create the sheet by wrapping the material around to form the closed loop. Thus, the degree of increase of the liquid pathways from one edge of the sheet to the other can be controlled.

The present invention has been described herein purely by way of example, and various additions, omissions and changes would be readily apparent to one skilled in the art, and therefore also fall within the scope and spirit of the invention. The terms comprise, comprising, comprises are, unless the context clearly demands otherwise, intended to be interpreted in the inclusive sense, that is, including, but not necessarily limited to.

1. A dispenser for dispensing volatile material by evaporation, the dispenser comprising:
a reservoir, for containing volatile material to be dispensed;
a dispensing screen for dispensing volatile material by evaporation, the dispensing screen comprising a sheet of material having continuous first and second edges and forming a continuous loop of material extending between the first and second edges; and
communicating means, for carrying volatile material from the reservoir to an in use upper edge of the dispensing screen, wherein the material of the dispensing means is acutely angled to an in use vertical axis of the dispenser.

2. A dispenser according to claim 1, wherein the dispensing screen is mounted around the reservoir.

3. A dispenser according to claim 1, wherein the dispensing screen is mounted concentric to the in use vertical axis of the reservoir.

4. A dispenser according to claim 1, wherein the communicating means comprises a wicking element, extending from inside the reservoir to the first edge of the dispersing screen.

5. A dispenser according to claim 1, wherein the communicating means comprises more than one wicking elements arranged between the reservoir and the screen, the wicking elements in communication with one another to deliver volatile material from the reservoir to the screen.

6. A dispenser according to claim 1, wherein the height of the dispensing screen is the same or less than the height of the reservoir.

7. A dispensing screen for dispensing, by evaporation, volatile materials applied thereto, the screen comprising:
a sheet of material; and
diverting means formed in or on the sheet,
wherein the diverting means form a minimum path length of the sheet for volatile material flowing along it between at least a portion of a first edge and an opposing second edge of the sheet, which minimum path length is longer than the shortest distance between the first and second edges along the surface of the sheet,
wherein the first and second edges of the sheet are each joined so that each is continuous, and the sheet is formed into a continuous loop of material between them,
and wherein a first closed notional ring on the surface of the material has a first length around its circumference and a second closed notional ring on the surface of the material, substantially parallel thereto and separated therefrom, has a second length around its circumference, the second length being longer than the first length.

8. A dispensing screen according to claim 7, wherein one edge is longer than the other.

9. A dispensing screen according to claim 8, wherein the second edge is longer than the first edge.

10. A dispensing screen according to claim 7, wherein at least one of the loops is situated between the first and second edges.

11. A dispensing screen according to claim 7, wherein the sheet is held in shape by a support at or adjacent each edge of the screen.

12. A dispensing screen according to claim 11, wherein the supports are substantially parallel.

13. A dispensing screen according to claim 7, wherein the sheet is formed of a woven material.

14. A dispensing screen according to claim 7, wherein the material is stretchable and resilient.

15. A dispensing screen according to claim 7, wherein the sheet of material is substantially conical or frustoconical.

16. A dispensing screen according to claim 7, wherein the sheet of material is tensioned.

17. A dispensing screen according to claim 7, wherein the diverting means comprises a plurality of fluid pathways, and the number of fluid pathways increases from the first edge to the second edge of the sheet.

18. (canceled)

19. A dispenser according to claim 1, wherein a further support is provided between the two edges, and parallel thereto, to hold a ring of the sheet to a larger minimum distance laterally from the notional axis than at least one of the edges.

20.-21. (canceled)
